(19) United States
(12) Patent Application Publication
(10) Pub. No.: US 2005/0061336 A1
(43) Pub. Date: Mar. 24, 2005

(54) Apparatus and method for serving medical device application content to a remote computing device

(76) Inventors: Steven M. Goetz, Brooklyn Center, MN (US); Duane Bourget, Albertville, MN (US); Lance Beall, Andover, MN (US); David W. Lee, Apple Valley, MN (US)

Correspondence Address: MEDTRONIC VASCULAR, INC.;IP LEGAL DEPARTMENT, 3576 UNOCAL PLACE, SANTA ROSA, CA, 95403, US

(21) Appl. No.: 10/949,627

(22) Filed: Sep. 23, 2004

Related U.S. Application Data
(60) Provisional application No. 60/505,522, filed on Sep. 24, 2003.
(60) Provisional application No. 60/589,993, filed on Jul. 20, 2004.

(51) Int. Cl. A61B 19/00

(52) U.S. Cl. 128/899; 128/903; 128/904

(57) ABSTRACT

A telemetry interface module according to the invention may be utilized to facilitate remote programming of a patient medical device such as an implantable medical device. The telemetry interface module includes processing logic, memory, and various software/firmware applications that are capable of generating a user interface (“UI”) description for transmission to a remote computing device. The UI description is transmitted in accordance with a standardized data communication protocol, and the remote computing device renders a UI in response to the received UI description. The remote computing device processes and renders the UI using its native operating system and native UI controls, thus eliminating the need for computing hardware customized for the particular medical device telemetry application.

### CROSS-REFERENCE TO RELATED APPLICATIONS

This application claims the benefit of U.S. provisional patent application Ser. No. 60/505,522, filed Sep. 24, 2003. This application also claims the benefit of U.S. provisional patent application Ser. No. 60/589,993, filed Jul. 20, 2004.

### TECHNICAL FIELD

The present invention generally relates to telemetry systems for implantable medical devices. More particularly, the present invention relates to a telemetry interface module for communicating data between an implantable medical device and a computing system.

### BACKGROUND

The prior art is replete with electronic and mechanical medical devices, suitable for use outside and inside the body, for treating a variety of patient conditions. Devices configured for use inside the body (i.e., implantable medical devices or IMDs) include devices such as neurostimulators, drug delivery devices, pacemakers, defibrillators, and cochlear implants. Clinicians use IMDs alone or in combination with therapeutic substance therapies and surgery to treat patient medical conditions. For some medical conditions, implantable medical devices provide the best, and sometimes the only, therapy to restore an individual to a more healthful condition and a fuller life.

IMDs are often used in conjunction with one or more computer and/or telecommunication systems and components. For example, an IMD may be designed to transmit to a computing system and/or receive from a computing system: data, programming instructions, energy for power supply replenishment, or the like. Information obtained from the IMD may be stored and subsequently transmitted to a physician or patient caregiver or a database on demand or automatically. Many ways of using the information are known, including decision making to provide optimum medical care to the person with the medical condition.

An IMD may be configured to communicate with an external device such as a computer-based programmer that facilitates physician or patient interaction with the IMD. A programmer may perform some or all of the following functions: create, store, and transfer stimulation therapy programs, where such programs govern the delivery of therapy to the patient; replenish a rechargeable power source in the IMD; monitor performance characteristics of the IMD; or generate a suitably formatted user interface for IMD or other data.

Traditionally, an IMD programming system includes three significant components. The first component, a telemetry head, communicates with the IMD via some form of wireless telemetry protocol. The second component is some form of computing device or system that supports the communication with the IMD and provides an interface for the user. The third component is one or more software applications that manage the programming process. The software application takes user inputs from the computing device, performs the necessary processing and logic, manages the communication with the IMD via the telemetry head, processes responses from the IMD, and displays appropriate information to the user via a user interface at the computing device. These three components are usually highly integrated - often physically connected together - and together form a device programmer.

In conventional systems, the bidirectional communication between the IMD and the devoted computing device is handled via a distinct telemetry head that is connected to the computing device. The telemetry head communicates with a compatible IMD telemetry element integrated into the IMD. The bi-directional telemetry communication between the IMD and the telemetry head is typically conduced at frequencies in a range from about 150 kHz to 200 kHz using existing telemetry protocols, i.e., an agreed-upon format for transmitting data between two devices.

Traditionally, the IMD manufacturer specifies, develops, and controls production of two of the three components discussed above: the telemetry head and the programming software application. The protocols and information processed by these components are usually proprietary, and are directly relevant to the functioning of the IMDs themselves. The third component, the computing device, which is necessary to support the functioning of the telemetry head and the software application, is not itself a core requirement for the functioning of the therapy and it often utilizes conventional computing hardware provided by computer equipment manufacturers. In practice, however, the computing device incorporated into a programmer accounts for much of the development effort, expense, and manufacturing effort associated with the programmer.

Accordingly, it would be desirable to have an IMD telemetry interface module that is decoupled from the related computing device, thus facilitating IMD telemetry while leveraging existing computing devices and platforms. Furthermore, other desirable features and characteristics of the invention will become apparent from the subsequent detailed description and the appended claims, taken in conjunction with the accompanying drawings and the foregoing technical field and background.

### BRIEF SUMMARY

A medical device telemetry interface module according to the invention communicates with a general purpose, existing computing device, system, or platform via standard communication protocols. In this manner, the interface module can: leverage existing consumer and/or industrial markets for general computing devices; support hardware-agnostic IMD programming and therapy software applications; leverage “off the shelf” computing hardware; minimize dependency on single-source or customized hardware components; and provide an environment for enhanced IMD data transfer, IMD data processing, remote network-based IMD programming, or remote network-based patient management.

The above and other aspects of the invention may be carried out in one form by a telemetry interface module including a telemetry head element configured to communicate with an IMD and receive data from the IMD, processing logic that generates a user interface description in response to the data, and a server application that provides the user interface description to at least one remote computing device.

### BRIEF DESCRIPTION OF THE DRAWINGS

A more complete understanding of the present invention may be derived by referring to the detailed description and claims when considered in conjunction with the following figures, wherein like reference numbers refer to similar elements throughout the figures.

FIG. 1 is a schematic representation of a telemetry interface module in communication with a variety of existing remote computing devices;

FIG. 2 is a schematic representation of a prior art IMD programming system;

FIG. 3 is a schematic representation of a telemetry interface module in communication with a remote computing device;

FIG. 4 is a schematic representation of an example network environment for a telemetry interface module;

FIG. 5 is a schematic representation of an example remote programming environment for a medical device;

FIG. 6 is a schematic representation of a telemetry interface module; and

FIGS. 7 and 8 are flow diagrams of an example medical device programming process according to the invention.

### DETAILED DESCRIPTION

The following detailed description is merely exemplary in nature and is not intended to limit the invention or the application and uses of the invention. Furthermore, there is no intention to be bound by any expressed or implied theory presented in the preceding technical field, background, brief summary or the following detailed description.

The invention may be described herein in terms of functional and/or logical block components and various processing steps. It should be appreciated that such block components may be realized by any number of hardware, software, and/or firmware components configured to perform the specified functions. For example, an embodiment of the invention may employ various integrated circuit components, e.g., memory elements, digital signal processing elements, logic elements, look-up tables, or the like, which may carry out a variety of functions under the control of one or more microprocessors or other control devices. In addition, those skilled in the art will appreciate that the present invention may be practiced in conjunction with any number of data transmission protocols and that the systems and protocols described herein represent specific examples for the invention.

For the sake of brevity, conventional techniques related to IMD telemetry, IMD data processing, data communication protocols, computer network architectures, user interface generation and manipulation, and other functional aspects of the systems (and the individual operating components of the systems) may not be described in detail herein. Furthermore, the connecting lines shown in the various figures contained herein are intended to represent example functional relationships and/or physical couplings between the various elements. It should be noted that many alternative or additional functional relationships or physical connections may be present in a practical embodiment.

Briefly, the invention relates to an apparatus and methods for leveraging the existing computing infrastructure of a clinical environment through the use of a telemetry interface module that incorporates the necessary application software and has the capability to serve the user interface (“UI”) for the application software to a remotely located general purpose computing platform. Such general purpose computing platforms are increasingly easy to find in the clinical environments in which device programming is accomplished.

In practice, the telemetry interface module may incorporate the necessary hardware, software, and/or firmware to run the communications link with an IMD. The module may also contain the processing logic necessary to generate and encode messages for the IMD and to process responses from the IMD. The module may further contain the processing logic or software necessary to generate UIs (e.g., screens, displays, or other content for rendering) for the user to manage the therapy. For example, a typical UI for an IMD application might allow a clinician to observe settings and status, form and execute new settings, perform diagnostics, or the like. The module need not contain the UI hardware itself, but it may include a minimal (inexpensive) UI. In the practical embodiment, the module generates a UI description, the UI description is provided to a remote computing platform, and the remote computing platform renders a UI based on the received UI description.

The means for such a system currently exist in the consumer market. For example, embedded web servers can be implemented in very modest hardware/software environments. One can envision a telemetry based programming system that serves its UI screens to a caregiver's laptop computer as HTML formatted pages over a conventional HTTP data communication link. A telemetry interface module configured in accordance with the invention could appear as just another node on an existing network—with its own IP address and/or other unique identification - and the user could connect to the telemetry interface module by entering its address into an appropriate, off-the-shelf browser application running on a standard personal computer. Other software technologies (both proprietary and standards based) may also be leveraged, such as Java applets, WAP interfaces, remote desktop tools, etc.

Connection issues (cabling, adaptors, and regulatory concerns related to electromagnetic isolation and the like) likely make the preferable physical interface for such a system wireless. A number of suitable wireless technologies already exist (e.g., the 802.11 family of standards, Bluetooth, GPRS). Alternately, the connection between the telemetry interface module and the remote computing device could be wired, using any appropriate standard such as Ethernet, IEEE 1394 (Firewire), or USB. Some wired protocols such as USB have the additional advantage of providing power as well as supporting data transfer.

A server-capable telemetry interface module as described herein may include several functional elements or components. Such functional elements may include a subsystem for communicating (via, e.g., a proprietary protocol) with the medical device, a processing element, a data and program memory element for storing application programs and associated data, and a communication interface for providing content to the remote host platform. The memory component may include removable storage media for the transfer of programs and/or data between modules.

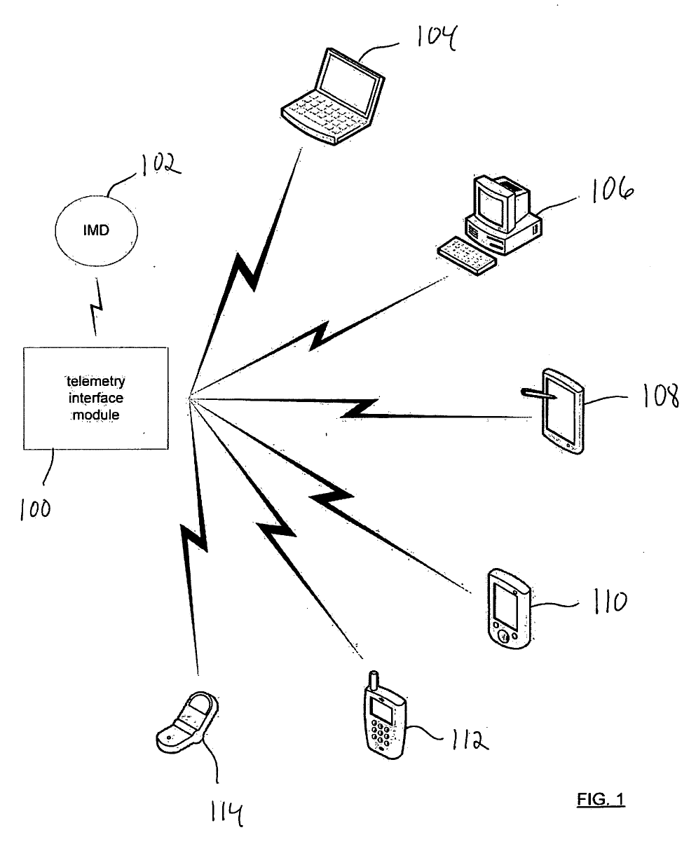

The general computing platforms that would be leveraged by such a system vary depending on the details of the interface. In one practical configuration that employs a wireless link to serve content to an industry standard browser application resident at the remote computing device, a large number of destination devices could be supported, including, without limitation: cell phones, personal digital assistants (“PDAs”), portable computers such as laptops and tablet PCs, or standard desktop PCs. In this regard, FIG. 1 is a schematic representation of a telemetry interface module 100 in communication with a variety of existing remote computing devices. For the sake of completeness, FIG. 1 depicts an IMD 102 in communication with module 100. As depicted in FIG. 1, a practical module 100 may be suitably configured to support wireless (and/or wired) communication with one or more of: a notebook computer 104, a desktop computer 106, a tablet computer 108, a PDA 110, a smart phone 112, a mobile phone 114, or the like. Depending on the chosen destination computing device, the system could fill one of several roles—a main clinician programmer with full features (laptop/tablet), a limited function or task specific programmer (PDA), or a patient programmer (PDA/phone). In each scenario, a telemetry interface module configured in accordance with the invention can leverage the existing host device hardware without having to install any proprietary applications or driver software on the host device.

This type of system has several advantages. First, decoupling the general computing platform from the telemetry interface module has development benefits. Technologies introduced into the consumer market are much easier to leverage, reducing the investment in their incorporation into proprietary platforms. Host platform hardware can be tailored to the needs of the therapy (for example, laptop or PC technologies for interface intensive applications, and PDA or cell phones for simpler needs). The core elements of the system can be easily reused across product lines and project cycles.

Second, this architecture provides distinct customer benefit. Programming solutions can be very low cost (leveraging the interface components already present in the clinical environment). In addition, the ergonomics of the telemetry link can be significantly improved. Currently, the need to see a display on a stand-alone programming device coupled with the short length of existing telemetry head cables can make the programming experience awkward. A decoupled system, especially one that uses a wireless link, removes these burdens.

Third, such an arrangement enables a number of advanced features. For example, if the programming system uses a clinician's office computer as the UI, exporting session report data to that computer from the programming system becomes trivial. Data could be saved in the internal memory of the telemetry interface module and then moved to the computer or other computing infrastructure via standard file transfer means, or the data could be directly saved from the browser environment to the hard drive of the host platform.

A telemetry interface that is both application aware and wireless network enabled makes remote programming possible (consider a follow-up consultation accomplished via an 802.11 WiFi hotspot in a coffee shop). The information stored in the medical device would be accessible from any compatible host platform in any environment, without the need for a live Internet or other network connection (where instead the necessary capability is incorporated into the telemetry interface module).

The telemetry interface module could also be designed to support multiple simultaneous sessions with multiple host computers. In complex or involved procedures, this would allow the tasks to be shared by multiple practitioners. For instance, one clinician might monitor uplinked sensor waveforms while a second clinician, using a second computing platform, updates or alters the parameters of the medical device. Conversely, a single host computer could connect to multiple telemetry interface modules. This could allow the direct transfer of data from one medical device to another, the establishment of multiple simultaneous sessions (in a patient with multiple devices, for instance), or the real time comparison of the operation of two or more medical devices.

Due to the regulatory environment, the complexity of the applications, and other practical factors, production of IMD programming systems is a challenging endeavor. By decoupling the core components of such a system from the supporting elements in such a way as to facilitate the reuse of the core components and the leveraging of existing computing infrastructures, the system described herein provides the opportunity to reduce the complexity of this task and increase the utility of the system for the end customers.

A variety of additional remote programming solutions may be possible with a telemetry interface module as described herein, including, without limitation: the provision of a remote programming or diagnostic station for patients patient in public places; pushing medical device data automatically to a database; continual medical device monitoring; wireless IMD data transfer to a computer, a network, a printer, or other off the shelf peripherals; enabling a patient monitor device to communicate directly to a database; enabling the medical device to automatically send an alarm through a cell phone to a caregiver or a hospital; or enabling an automatic dialer to contact the remote medical device (either an implanted device or an external device).

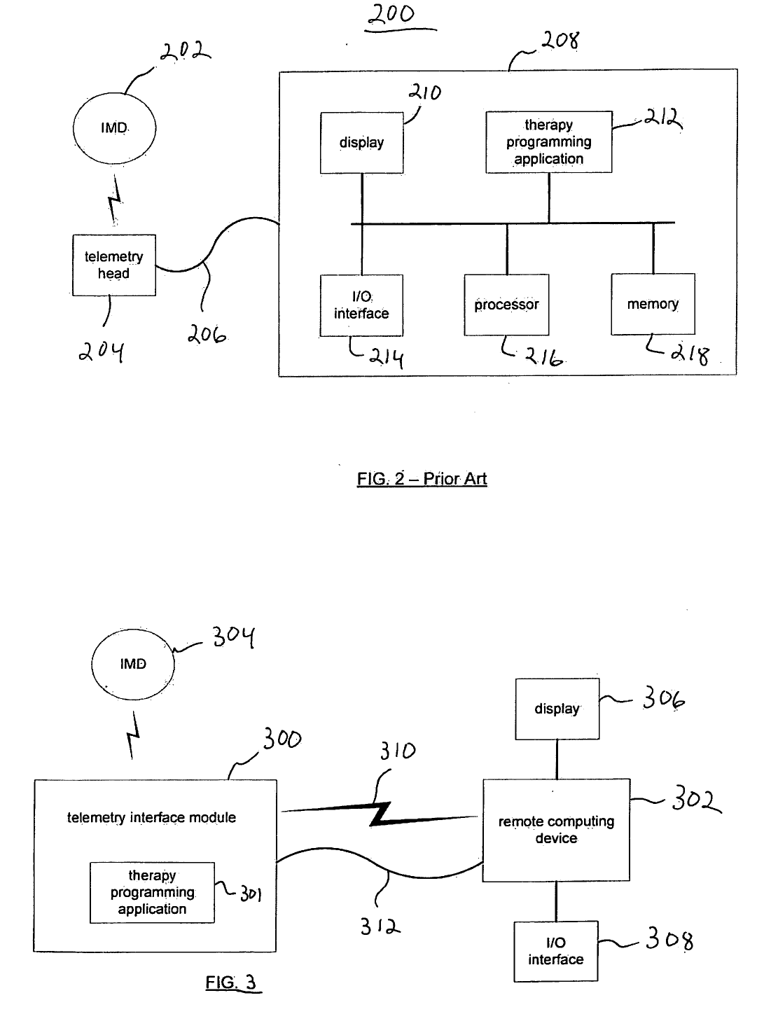

As mentioned above, a telemetry interface module according to the invention can communicate with existing computing devices (see FIG. 1) and leverage the existing computing device hardware to reduce the hardware-dependence of prior art programming systems. FIG. 2 is a schematic representation of a typical prior art IMD programming system 200. System 200 is a stand-alone apparatus that is customized according to the particular IMD 202 and according to the programming and diagnostic requirements of IMD 202. Generally, system 200 includes a telemetry head 204 attached (via a cable 206) to a housing 208. Telemetry head 204 and housing 208 form a single unit commonly referred to as a programmer.

System 200 includes a display 210, a therapy programming application 212, an input/output interface 214, a processor 216, and a memory element 218. Although not shown in FIG. 2, system 200 may include additional hardware, software, firmware, or functional elements that are traditionally associated with general purpose computing devices. In practice, display 210 renders a UI to the user of system 200 to facilitate viewing of IMD data, diagnostics, and programming of IMD 202. Input/output interface 214 allows a caregiver to manipulate the UI to update or alter the therapy program associated with IMD 202. Therapy programming application 212 governs the communication with IMD 202, the generation of the UI, and the updating of the therapy program for IMD 202. Notably, system 200 is stand-alone in that all of the IMD data processing, UI generation and rendering, and IMD programming procedures are performed by system 200 alone.

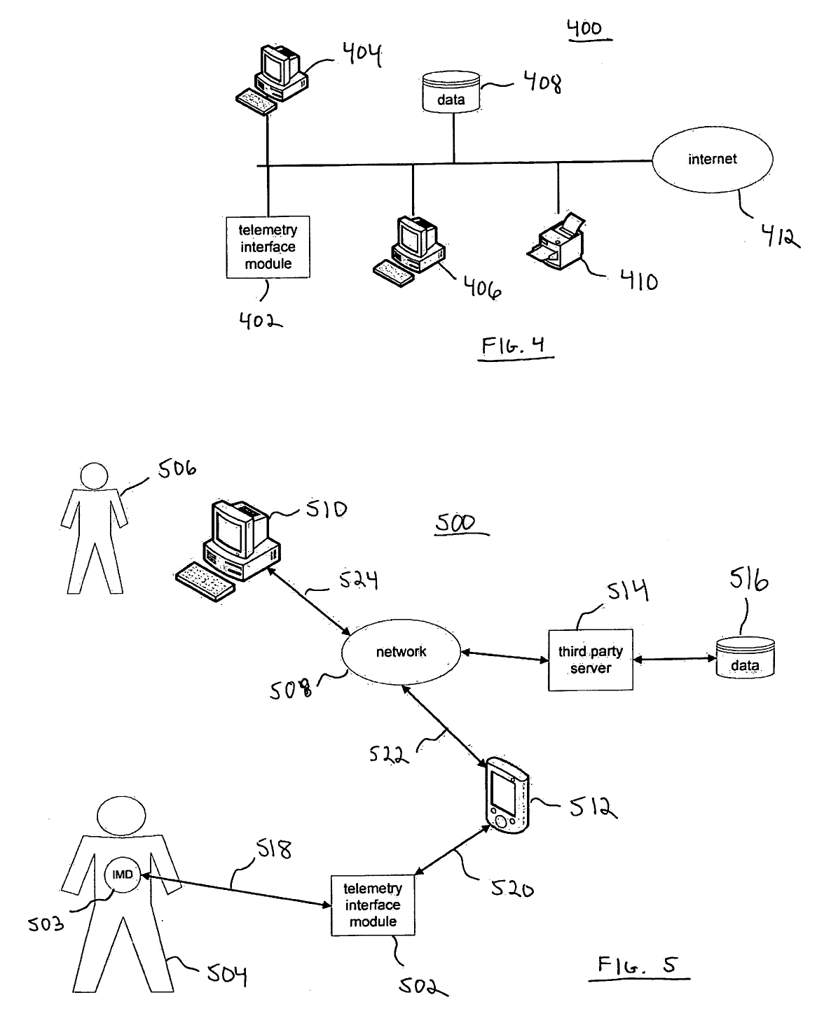

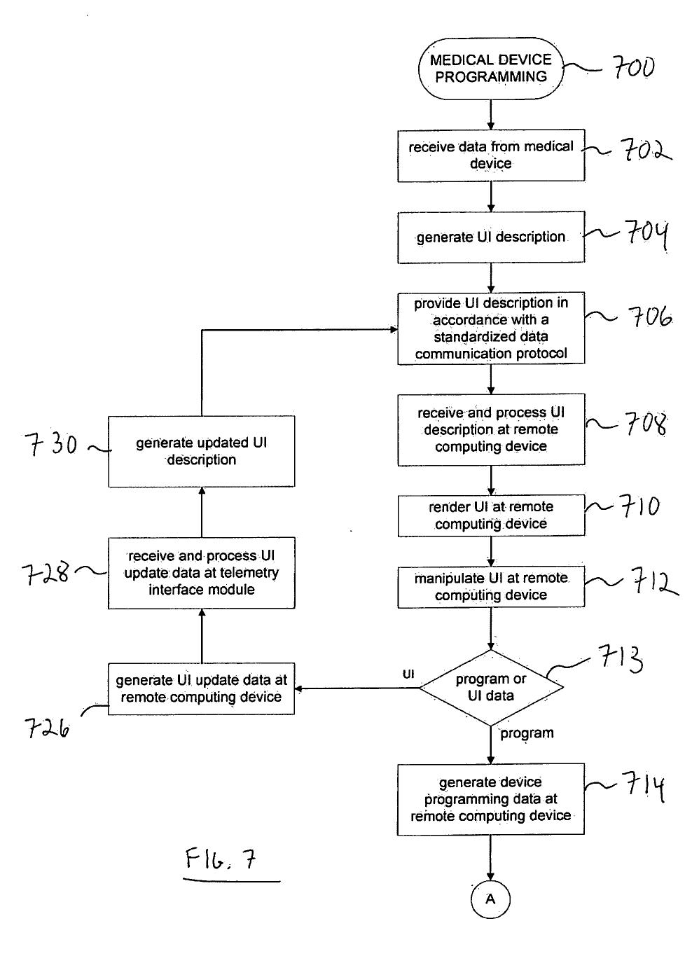

In contrast to IMD programming system 200, a telemetry interface module 300 according to the invention is generally depicted in FIG. 3. FIG. 3 is a schematic representation of telemetry interface module 300 in communication with a remote computing device 302. In the example embodiment, telemetry interface module 300 is configured to support bi-directional communication with a medical device, such as an IMD 304, using existing technologies. Thus, telemetry interface module 300 can be suitably configured to support legacy IMDs that may be already implanted in patients. Notably, and as described in more detail below in connection with FIG. 6, telemetry interface module 300 includes a therapy programming application 301 that supports programming of IMD 304, and supports the generation of UI descriptions for remote computing device 302.

Remote computing device 302 includes a display element 306 and an input/output interface 308. In the practical embodiment, remote computing device 302 need not be customized, altered, or loaded with proprietary software in order to communicate with and support the functions of telemetry interface module 300. Accordingly, the specific configuration, operating characteristics, and functionality of display element 306 and input/output interface 308 can vary depending upon the practical implementation of remote computing device 302. For example, if remote computing device 302 is a desktop computer, then display element 306 may be a CRT, LCD, or plasma monitor, and input/output interface 308 may include a keyboard and a pointing device such as a mouse or touchpad (interface 308 may also include a speaker system, a microphone system, a camera system, or the like). Alternatively, if remote computing device 302 is a PDA, then display element 306 may be a small scale LCD integrated into the PDA itself, and input/output interface 308 may include a small scale keypad, a stylus writing screen, a touchpad, or the like.

Telemetry interface module 300 is suitably configured to support bi-directional data communication with remote computing device 302. Such data communication may be carried out over any number of wireless data communication links 310 and/or any number of wired data communication links 312. In one preferred embodiment, a wireless data communication link 310 serves as the primary communication link, and wired data communication link 312 serves as a secondary or backup communication link. Wired data communication link 312 may be desirable in environments susceptible to electromagnetic interference or in environments that are otherwise not particularly suitable for wireless data communications.

FIG. 4 is a schematic representation of an example network environment 400 for a telemetry interface module 402. In accordance with one practical implementation of the invention, telemetry interface module 402 serves as a node in network environment 400. This architecture is feasible because telemetry interface module 402 is suitably configured to support conventional and standardized data communication protocols that are commonly utilized in the context of computer networks. For example, telemetry interface module 402 may be configured to support one or more of the following known data communication techniques (without limitation): Bluetooth; IEEE 802.11 (any variation thereof); Ethernet; IEEE 1394 (Firewire); GPRS; USB; IEEE 802.15.4 (ZigBee); or IrDA (infrared). Once connected to network environment 400, telemetry interface module 402 may communicate with other network components, e.g., computers 404/406, a network server or database 408, a printer 410, or other peripherals. Computer 404 or computer 406 may serve as a remote computing device as discussed above in connection with FIG. 3. Furthermore, once connected to network environment 400, telemetry interface module 402 may have access to one or more other networks, such as the Internet 412. Internet connectivity facilitates bi-directional communication between telemetry interface module 402 and a virtually limitless number of remote computing devices.

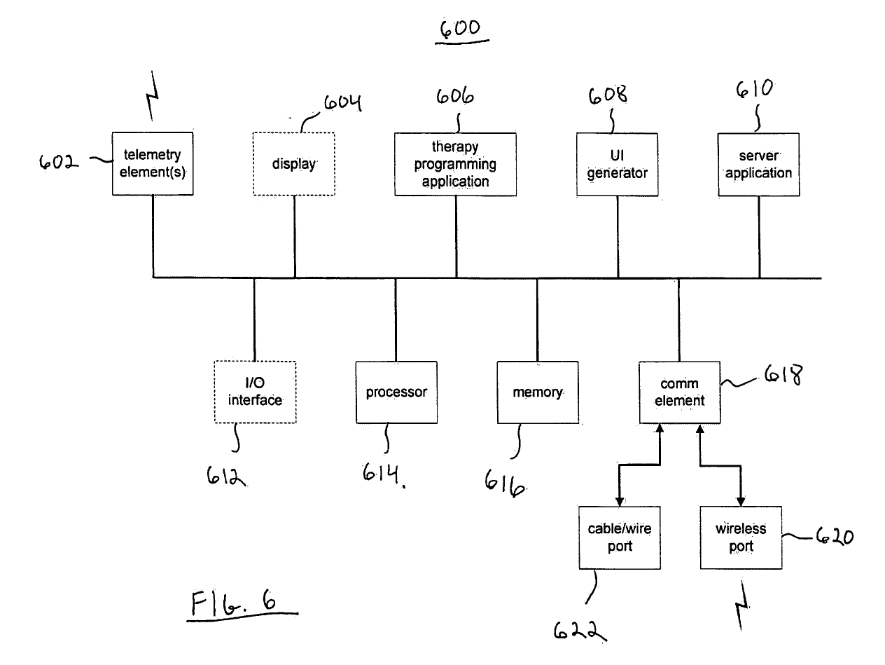

FIG. 5 is a schematic representation of an example remote programming environment 500 that includes a telemetry interface module 502. Remote programming environment 500 supports remote programming of a medical device such as an IMD 503. Although an IMD application is depicted in FIG. 5, the remote programming concept may also apply to the programming of an external medical device associated with a patient 504. Furthermore, the remote programming concept may apply to the concurrent or serial programming of any number of different medical devices. Briefly, remote programming environment 500 facilitates remote programming of IMD 503 by a caregiver 506 via a network 508.

Remote programming environment 500 generally includes a remote computing device 510, telemetry interface module 502, IMD 503, network 508, an optional patient computing device 512, an optional third party server 514, and an optional database or repository 516 associated with server 514. The various components in remote programming environment 500 may communicate with each other using one or more standardized or proprietary data communication protocols and technologies. For example, IMD 503 may communicate with telemetry interface module 502 via a wireless link 518 that conveys data in accordance with a proprietary protocol. In practice, wireless link 518 employs magnetic inductive coupling between IMD 503 and telemetry interface module 502 to communicate encoded data originating at IMD 503 and to communicate programming commands originating at telemetry interface module 502. Telemetry interface module 502 may communicate with patient computing device 512 via a wireless link 520 that conveys data in accordance with a standardized protocol. For example, wireless link 520 may employ (without limitation) Bluetooth, IEEE 802.11, or infrared data communication protocols. Patient computing device 512 may communicate with network 508 (e.g., the Internet, a WAN, or a LAN) via a wireless link 522 configured in accordance with a standardized data communication protocol such as GPRS. In one preferred practical embodiment, network 508 is the Internet and remote programming environment 500 supports programming of IMD 503 from any remote location having a suitable Internet connection or communication link 524.

As described in more detail below, telemetry interface module 502 may be configured to receive patient-related data from IMD 503, generate a UI description that conveys the patient-related data in a meaningful context, and provide the UI description for rendering at remote computing device 510. In this regard, remote computing device 510 is capable of rendering a UI, determined at least in part by telemetry interface module 502, for viewing data related to IMD 503. Caregiver 506 can then view the UI to obtain information related to the operation of IMD 503, and, if necessary, manipulate the UI to update the IMD program (i.e., change one or more operating parameters of IMD 503) and/or to update the UI description generated by telemetry interface module 502. In a practical deployment, the UI is one or more web pages that can be rendered with a standard web browser application on remote computing device 510. The programming data is sent back to IMD 503 via network 508 and telemetry interface module 502.

The optional patient computing device 512 may serve as a liaison between remote computing device 510 and telemetry interface module 502. For example, patient computing device 512 may provide a UI display, a keypad, indicator lights, and/or control buttons for ease of use by patient 504 (this option may be desirable if telemetry interface module 502 includes limited UI capabilities). Patient computing device 512 may be configured to download and store the programming data from remote computing device 510 such that the programming data can be transferred to IMD 503 at a time that is convenient for patient 504. The updated therapy prescription may be downloaded to and stored by patient programmer 512 in advance or dynamically in real time. In one example embodiment, patient 504 may be paged via patient computing device 512 in response to the updating of the IMD program at remote computing device 510. The paging may utilize simple text messaging (“SMS”) techniques or any known communication technique. The patient 504 may be prompted with a “Program Device” message and instructed to position telemetry interface module 502 in a suitable location for programming. Thereafter, the updated IMD program may be transferred from patient computing device 512 to IMD 503 via telemetry interface module 502. Alternatively, the IMD program may be transferred from patient computing device 512 for storage at telemetry interface module 502, thus enabling subsequent programming of IMD 503 from telemetry interface module 502. A similar procedure can be performed to support a deployment that does not include patient computing device 512—the updated programming data may be transferred directly from remote computing device 510 to telemetry interface module 502, and the actual programming of IMD 503 could be performed dynamically or after the updated program has been completely transferred to telemetry interface module 502.

The optional third party server 514 and database 516 may serve as a data repository for archiving, monitoring, or regulatory purposes. In such an implementation, remote computing device 510 would communicate the IMD programming information via third party server 514. In an alternate embodiment, third party server 514 provides the functionality of a telemetry interface module (as described in more detail below), thus enabling the use of a less intelligent telemetry interface module in connection with a server-based network deployment.

Telemetry interface module 502 is also capable of supporting a remote programming procedure that is initiated by caregiver 506 rather than patient 504 or telemetry interface module 502. In accordance with this alternate procedure, caregiver 506 initially logs into an appropriate web site or portal using remote computing device 510. The portal may be maintained by third party server 514 or any suitable server, and access to the portal may be protected using conventional security and authentication processes. An appropriate UI is rendered at remote computing device 510, and that UI allows caregiver 506 to identify patient 504 and IMD 503. The UI also allows caregiver 506 to update the IMD therapy program. In response to a program update, the system pages patient 504 via patient computing device 512 or telemetry interface module 502. Thereafter, the IMD programming can be carried out as described above.

FIG. 6 is a schematic representation of a telemetry interface module 600 configured in accordance with the invention. Telemetry interface module 600, or an equivalent architecture, may be utilized in connection with the various deployments described above in connection with FIGS. 1, 3, 4, and 5. Telemetry interface module 600 generally includes one or more telemetry elements 602, an optional display element 604, a therapy programming application 606, a UI generator 608, a server application 610, an optional input/output interface 612, a processor or controller 614, memory 616, and a communication element 618. Telemetry interface module 600 may also include a number of conventional hardware, software, firmware, or logical elements found in general purpose computing architectures (not shown). These elements may be coupled together or otherwise able to communicate with each other as needed to support the functionality of telemetry interface module 600. In an actual deployment, therapy programming application 606, UI generator 608, server application 610, and communication element 618 (or portions thereof) are realized as processing logic or logical elements, and such processing logic may be realized as one or more pieces of software/firmware. For example, UI generator 608, server application 610, and communication element 618 may be implemented in therapy programming application 606 itself. Furthermore, therapy programming application 606 may include a suitable operating system for telemetry interface module 600.

A “server” is often defined as a computing device or system configured to perform any number of functions and operations associated with the management, processing, storage, retrieval, and/or delivery of data, particularly in a network environment. Alternatively, a “server” or “server application” may refer to software or firmware that performs such processes, methods, and/or techniques, and server application 610 functions in this manner. As in most commercially available general purpose computing devices, a practical computing architecture that supports telemetry interface module 600 may be configured to run on any suitable operating system such as Unix, Linux, the Apple Macintosh OS, any variant of Microsoft Windows, a commercially available real time operating system, or a customized operating system, and it may employ any number of processors 614, e.g., the Pentium family of processors by Intel, the processor devices commercially available from Advanced Micro Devices, IBM, Sun Microsystems, or Motorola, or other commercially available embedded microprocessors or microcontrollers.

With regard to telemetry interface module 600 (and the remote computing devices discussed herein), the respective servers and other logical elements may communicate with system memory (e.g., a suitable amount of random access memory), and an appropriate amount of storage or “permanent” memory. For telemetry interface module 600, memory 616 may represent such random access memory and/or such permanent memory. The permanent memory may include one or more hard disks, floppy disks, CD-ROM, DVD-ROM, magnetic tape, removable media, solid state memory devices, or combinations thereof. In accordance with known techniques, the operating system programs and the server application programs reside in the permanent memory and portions thereof may be loaded into the system memory during operation. In accordance with the practices of persons skilled in the art of computer programming, the invention is described herein with reference to symbolic representations of operations that may be performed by the various computing components or devices. Such operations are sometimes referred to as being computer-executed, computerized, software-implemented, or computer-implemented. It will be appreciated that operations that are symbolically represented include the manipulation by the various microprocessor devices of electrical signals representing data bits at memory locations in the system memory, as well as other processing of signals. The memory locations where data bits are maintained are physical locations that have particular electrical, magnetic, optical, or organic properties corresponding to the data bits.

When implemented in software or firmware, various elements of the systems described herein (which may reside at telemetry interface module 600, the remote computing devices, or elsewhere in the network environment) are essentially the code segments or instructions that perform the various tasks. The program or code segments can be stored in a processor-readable medium or transmitted by a computer data signal embodied in a carrier wave over a transmission medium or communication path. The “processor-readable medium” or “machine-readable medium” may include any medium that can store or transfer information. Examples of the processor-readable medium include an electronic circuit, a semiconductor memory device, a ROM, a flash memory, an erasable ROM (EROM), a floppy diskette, a CD-ROM, an optical disk, a hard disk, a fiber optic medium, a radio frequency (RF) link, or the like. The computer data signal may include any signal that can propagate over a transmission medium such as electronic network channels, optical fibers, air, electromagnetic paths, or RF links. The code segments may be downloaded via computer networks such as the Internet, an intranet, a LAN, or the like.

Referring again to FIG. 6, telemetry interface module 600 includes at least one telemetry element 602, which is configured to communicate with a medical device to receive data from the medical device. Telemetry element 602 may also be configured to provide or transmit programming commands and/or other data to the medical device. In this regard, telemetry element 602, and any corresponding logical elements, individually or in combination, are example means for receiving medical device data, and example means for providing programming commands. As used herein, “medical device data” means any data or information that originates from the particular medical device, including, without limitation: register values corresponding to the status of the medical device, patient diagnostic data detected by the medical device, diagnostics of the device functionality (e.g., battery level or remaining life), a usage log of time stamped events, or historical status trends. In the example embodiment, telemetry element 602 is configured for wireless RF communication with an IMD using known techniques (such as inductive coupling) and known protocols (such as specific encoding protocols), which may or may not be proprietary. Telemetry interface module 600 may utilize multiple telemetry elements 602 to support a plurality of medical devices, thus facilitating concurrent programming of more than one medical device. The hardware configuration of the multiple telemetry elements 602 may be the same or different depending upon the practical application. Telemetry hardware, telemetry protocols, and the manner in which IMDs support bidirectional data communication are known to those skilled in the art and, therefore, will not be described in detail herein.

Telemetry interface module 600 may include an optional display element 604 that conveys visual information to the user under the control of processor 614 and therapy programming application 606. In practice, display element 604 may include a relatively small LCD screen, a few indicator lights, or the like. Likewise, telemetry interface module 600 may include an optional input/output interface 612 that accommodates user inputs and/or conveys audible or tactile information to the user under the control of processor 614 and therapy programming application 606. In practice, input/output interface 612 may include a few buttons, switches, an audio transducer, or the like. Display element 604 and input/output interface 612 may guide the user in the operation of telemetry interface module 600 and the programming of the medical device, and may communicate diagnostic data or IMD data to the patient. In some applications it may be desirable to keep telemetry interface module 600 compact, lightweight, inexpensive, and simple to use—such applications may employ a simple display element 604 (or no display element 604) and a simple input/output interface 612 (or no input/output interface 612).

Therapy programming application 606 represents the primary application software and/or firmware that governs the operation of telemetry interface module 600. Application 606 may perform, control, or govern the following and possibly other functions: decoding of the raw data obtained from the medical device; encoding data for transmission to the medical device via telemetry element 602; generating UIs; controlling printing of reports, data sheets, or the like; controlling UI rendering on display 604 (if applicable); formatting data for transmission in accordance with the supported data communication protocols between telemetry interface module 600 and the remote computing devices; processing programming data for the medical device; generating programming commands for the medical device; performing calculations on the processed device data; storing the processed device data for future use; and detecting the validity of the device data. As mentioned above, therapy programming application 606 may include, communicate with, or otherwise be associated with UI generator 608, server application 610, or communication element 618. These functional components are depicted as separate entities for convenience and to provide a better understanding of the invention.

UI generator 608 may be realized as processing logic configured to generate a UI description in response to data received from a medical device, e.g., an IMD. UI generator 608 may also be configured to generate updated UI descriptions in response to UI update data received from a remote computing device, where such UI update data may be generated by user manipulation of the UI rendered by the remote computing device. In this regard, UI generator 608, therapy programming application 606, and any corresponding logical or software elements, individually or in combination, are example means for generating a UI description. As used herein, a “UI description” means data or information that defines the layout, content, and configuration of a UI to be rendered by a suitable computing device, e.g., a remote computing device as described herein. The UI description may represent a UI template or form retrieved from memory 616 and populated with the medical device data or information derived from the medical device data. In addition, the UI description may be associated with a set of rules or algorithms stored in memory 616, a set of rules or algorithms stored in the medical device itself, or a set of dynamic rules or algorithms that may be influenced by user preferences, programming history, or other characteristics of the telemetry programming system.

In practice, the UI description may contain or define one or more text-based markup language files, such as an XML file, an HTML file, a web page, a MathML file, an SMIL file, an SGML file, or the like. In the example embodiment, the UI description corresponds to a web page that may have user-accessible controls or other dynamic content, along with text, images, waveforms, or other static content. The UI description may define standard file formats such as a spreadsheet file, a word processor file, a PDF file, or the like. In addition, the UI description may define or contain one or more served applications, such as Java applets, that can be transferred for execution by the remote computing device, possibly with ongoing control by telemetry interface module 600.

UI generator 608 may generate the UI description in response to operating characteristics of the remote computing device. This capability may be desirable to enable telemetry interface module 600 to communicate with a plurality of host computing devices having different configurations. For example, UI generator 608 may be suitably configured to generate the UI description for compatibility with the operating system of the given remote computing device (for example, the Windows OS, the Palm OS, the Windows Pocket PC OS, or the like). In this regard, it may be necessary for telemetry interface module 600 to obtain information regarding the configuration of the remote computing device for purposes of UI generation. This information may be transferred to telemetry interface module 600 during an initialization or pre-programming procedure, or in connection with a query exchange with the remote computing device upon establishment of a communication session. This feature allows telemetry interface module 600 to switch its output configuration mode according to the requirements of the particular remote computing device.

Server application 610 is configured to provide the generated UI description to at least one remote computing device for rendering by the remote computing device. It should be appreciated that server application 610, therapy programming application 606, and any corresponding logical or software elements, individually or in combination, are example means for providing the UI description to the remote computing device. In the example embodiment, server application 610 is realized as a standard web server that provides HTTP/HTML web pages. Alternatively (or additionally), server application 610 may be a file server that provides standard computer files via File Transfer Protocol, or an application server that provides application files such as Java applets, CGI scripts, or the like. Generally, server application 610 may be suitably configured to support any number of UI description architectures and server application 610 may leverage any number of conventional server technologies.

In a practical embodiment, the remote computing device receives and processes the UI description, and renders a suitable UI in response to the received UI description. In the preferred embodiment, the remote computing device renders the UI using its native operating system and the native UI controls associated with the native operating system. In other words, the telemetry programming system need not rely on any customization at the remote computing device.

Communication element 618, which communicates with server application 610, is suitably configured to communicate the UI description to the remote computing device in accordance with at least one data communication protocol. In the example embodiment, communication element 618 communicates with the remote computing device in accordance with at least one standardized data communication protocol (either wireless or wired). Such standardized data communication protocols include, without limitation: Bluetooth; IEEE 802.11 (any variation thereof); Ethernet; IEEE 1394 (Firewire); GPRS; USB; IEEE 802.15.4 (ZigBee); or IrDA (infrared). Communication element 618 may be realized with hardware, software, and/or firmware using known techniques and technologies. For example, telemetry interface module 600 may include a wireless port 620 configured to support bi-directional wireless data communication with the remote computing device and/or a cable or wire port 622 configured to support bi-directional data communication, via a tangible link, with the remote computing device. In this regard, communication element 618, wireless port 620, cable port 622, therapy programming application 606, and any corresponding logical or software elements, individually or in combination, are example means for providing the UI description to the remote computing device.

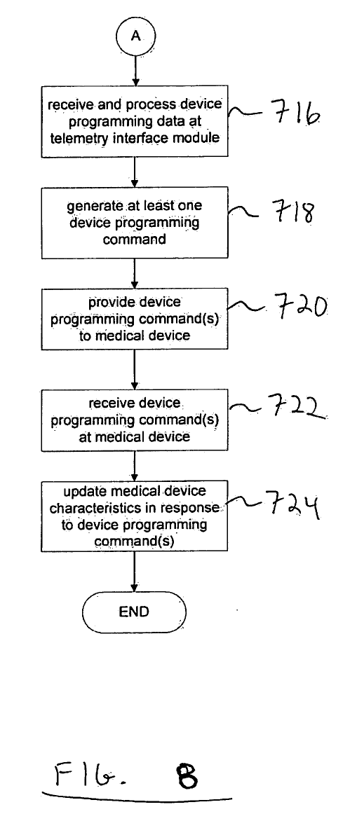

FIGS. 7 and 8 are flow diagrams of an example medical device programming process 700 that may be performed with the assistance of a telemetry interface module. The various tasks performed in connection with process 700 may be performed by software, hardware, firmware, or any combination thereof. For illustrative purposes; the following description of process 700 may refer to elements mentioned above in connection with FIGS. 1 and 3-6. In practical embodiments, portions of process 700 may be performed by different elements of the telemetry programming system, e.g., the medical device, the telemetry interface module, or the remote computing device. It should be appreciated that process 700 may include any number of additional or alternative tasks, the tasks shown in FIGS. 7 and 8 need not be performed in the illustrated order, and process 700 may be incorporated into a more comprehensive procedure or process having additional functionality not described in detail herein.

Medical device programming process 700 may begin by receiving data from one or more medical devices (task 702). In the example embodiment, task 702 is performed by the telemetry interface module and the data represents raw data provided by an IMD. In response to the received data, process 700 generates a UI description (task 704) that defines, determines, or otherwise specifies the configuration, format, layout, and content of a UI for a remote computing device. In the example embodiment, task 704 is performed by the telemetry interface module in connection with the processing of the received medical device data. Thereafter, process 700 provides the UI description to at least one remote computing device (task 706). In the preferred embodiment, task 706 is performed by the telemetry interface module and the UI description is provided in accordance with at least one standardized data communication protocol.

Unless transmission errors or a failed communication link adversely impact the delivery of the UI description, medical device programming process 700 performs a task 708. Task 708 receives and processes the UI description at the target remote computing device. Thus, in the practical embodiment, the remote computing device carries out task 708. Preferably, the native operating system of the remote computing device processes the UI description and uses native controls to render an appropriate UI at the remote computing device (task 710); the UI is rendered in response to the received UI description. The UI may be dynamic in nature to facilitate remote programming by a caregiver having access to the remote computing device. Remote programming or updating of the currently rendered UI may be accomplished via manipulation of the UI at the remote computing device (task 712). For example, the caregiver may change certain programmable operating characteristics or parameters of the IMD that are displayed in the UI, or the caregiver may change the appearance of the UI via normal interaction with the UI.

If the manipulation of the remote UI is associated with the generation of programming data, then medical device programming process 700 may proceed to a task 714. If, however, the manipulation of the remote UI results in the generation of UI update data, then process 700 may proceed to a task 726 (see below description).

The manipulation of the remote UI may result in the generation of device programming data at the remote computing device (task 714). In one embodiment, the device programming data can be realized as alphanumeric characters contained in a suitable markup language file packaged for routing back to the telemetry interface module or the patient computing device (if applicable). The programming data may be generated and sent dynamically in real time, or it may be generated and stored for batch transmission triggered by an event, e.g., after the caregiver confirms (using the UI) that the device programming is complete. Eventually, the telemetry interface module receives and processes the device programming data (task 716). The programming data serves as input data to the telemetry interface module, where such input data is received in response to the manipulation of the UI. In practice, the programming data may be received by communication element 618 via wireless port 620 and/or cable port 622. Thus, communication element 618, wireless port 620, cable port 622, therapy programming application 606, and any corresponding logical or software elements, individually or in combination, are example means for receiving input data in response to manipulation of the UI.

The received device programming data may be processed by therapy programming application 606 to generate at least one corresponding device programming command for the medical device (task 718). As used herein, a “device programming command” represents an instruction, information, a control, or data that is formatted in accordance with a data communication protocol supported between the medical device and the telemetry interface module. Device programming commands can be received and understood by the target medical device. For example, a device programming command may be realized as an encoded stream of bits that represent one or more IMD register values. It should be appreciated that therapy programming application 606 and any corresponding logical or software elements, individually or in combination, are example means for generating programming commands for the medical device.

Medical device programming process 700 continues by providing the device programming command or commands to the medical device (task 720). As mentioned above, the commands may be communicated using inductive RF techniques and in accordance with a proprietary encoded protocol. In a practical embodiment, task 720 may be performed by telemetry element 602 under the control of therapy programming application 606. Accordingly, telemetry element 606, therapy programming application 606, and any corresponding logical or software elements, individually or in combination, are example means for providing at least one programming command to the medical device.

In response to task 720, the medical device receives the programming commands (task 722) and updates its operating characteristics in response to the programming commands (task 724). In the preferred embodiment, the receiving of programming commands and the updating of the medical device registers is carried out in a conventional manner. The telemetry interface module may be further configured to communicate information back to the remote computing device (and/or to the patient computing device) to indicate successful updating of the medical device. Once the device has been successfully programmed, process 700 ends. Notably, multiple instantiations of process 700 may be concurrently executed to support remote programming or diagnostic reading of more than one medical device implanted in, carried on, or attached to the patient. Furthermore, process 700 may be repeated to accomplish iterative programming of a single medical device.

The manipulation of the remote UI may result in the generation of UI update data at the remote computing device (task 726). In one embodiment, the UI update data can be realized as alphanumeric characters contained in a suitable markup language file packaged for routing back to the telemetry interface module. The UI update data is preferably generated and sent dynamically in real time to facilitate immediate updating of the UI. Eventually, the telemetry interface module receives and processes the UI update data (task 728). The UI update data serves as input data to the telemetry interface module, where such input data is received in response to the manipulation of the UI. In practice, the UI update data may be received by communication element 618 via wireless port 620 and/or cable port 622.

In response to the UI update data, the telemetry interface module generates an updated UI description (task 730) that defines, determines, or otherwise specifies the configuration, format, layout, and content of an updated UI for the remote computing device. Thereafter, process 700 may be re-entered at task 706 to provide the updated UI description to the remote computing device for rendering as described above. Thus, the remote computing device can render and update its UI with the assistance of the telemetry interface module in an ongoing manner with or without the generation of programming data for the medical device.

While at least one practical embodiment has been presented in the foregoing detailed description, it should be appreciated that a vast number of variations exist. It should also be appreciated that the example embodiment or embodiments are not intended to limit the scope, applicability, or configuration of the invention in any way. Rather, the foregoing detailed description will provide those skilled in the art with a convenient road map for implementing the exemplary embodiment or exemplary embodiments. It should be understood that various changes can be made in the function and arrangement of elements without departing from the scope of the invention as set forth in the appended claims and the legal equivalents thereof.

1. A method for communicating information between a data source and a destination computer system, said method comprising:
receiving data at a telemetry interface module;
said telemetry interface module generating a user interface description in response to said data; and
said telemetry interface module providing said user interface description to a remote computing device.

2. A method according to claim 1, wherein said telemetry interface module generates said user interface description in response to operating characteristics of said remote computing device.

3. A method according to claim 1, wherein said telemetry interface module generates said user interface description for compatibility with an operating system of said remote computing device.

4. A method according to claim 1, wherein said data is received from an implantable medical device.

5. A method according to claim 1, wherein said data is received from an external patient device.

6. A method according to claim 1, wherein said telemetry interface module provides said user interface description in accordance with at least one standardized data communication protocol.

7. A method according to claim 1, wherein said user interface description contains information derived from said data.

8. A method according to claim 1, further comprising:
said remote computing device receiving said user interface description; and
said remote computing device rendering a user interface in response to said user interface description.

9. A method according to claim, 8, wherein said data is received from an implantable medical device (“IMD”), said method further comprising:
said telemetry interface module receiving input data in response to manipulation of said user interface; and
said telemetry interface module generating, in response to said input data, at least one programming command for said IMD.

10. A method according to claim 9, further comprising said telemetry interface module providing said at least one programming command to said IMD.

11. A method according to claim 8, wherein said user interface is a markup language file.

12. A method according to claim 8, wherein said data is received from an implantable medical device (“IMD”), said method further comprising:
said telemetry interface module receiving input data in response to manipulation of said user interface; and
said telemetry interface module generating, in response to said input data, an updated user interface description for said remote computing device.

13. A telemetry interface module comprising:
means for receiving medical device data;
means for generating a user interface description in response to said medical device data; and
means for providing said user interface description to a remote computing device.

14. A telemetry interface module according to claim 13, wherein said medical device data is received from an implantable medical device (“IMD”), said telemetry interface module further comprising:
means for receiving input data in response to manipulation of a user interface rendered by said remote computing device; and
means for generating, in response to said input data, at least one programming command for said IMD.

15. A telemetry interface module according to claim 14, further comprising means for providing said at least one programming command to said IMD.

16. A computer program architecture for facilitating communication of information between a data source and a destination computer system, said computer program architecture being embodied on computer-readable media, said computer program architecture having computer-executable instructions comprising:
instructions for receiving and processing medical device data;
instructions for generating a user interface description in response to said medical device data; and
instructions for providing said user interface description to a remote computing device.

17. A computer program architecture according to claim 16, wherein said medical device data is received from an implantable medical device (“IMD”), said computer program architecture further comprising:
instructions for receiving input data in response to manipulation of a user interface rendered by said remote computing device; and
instructions for generating, in response to said input data, at least one programming command for said IMD.

18. A computer program architecture according to claim 17, further comprising instructions for providing said at least one programming command to said IMD.

19. A telemetry interface module comprising:
a telemetry element configured to communicate with an implantable medical device (“IMD”) and to receive data from said IMD;
processing logic in communication with said telemetry element, said processing logic being configured to generate a user interface description in response to said data; and
a server application in communication with said processing logic, said server application being configured to provide said user interface description to at least one remote computing device.

20. A telemetry interface module according to claim 19, further comprising a communication element in communication with said server application, said communication element being configured to communicate said user interface description to said at least one remote computing device in accordance with at least one data communication protocol.

21. A telemetry interface module according to claim 20, wherein said communication element is configured to communicate said user interface description in accordance with at least one standardized data communication protocol.

22. A telemetry interface module according to claim 20, wherein said at least one data communication protocol comprises a wireless data communication protocol.

23. A telemetry interface module according to claim 19, wherein said server application comprises a web server.

24. A telemetry interface module according to claim 19, wherein said server application comprises a file server.

25. A telemetry interface module according to claim 19, wherein said server application comprises an application server.

26. A telemetry interface module according to claim 19, wherein said processing logic is configured to generate said user interface description in response to operating characteristics of said at least one remote computing device.

27. A telemetry interface module according to claim 19, wherein said processing logic is configured to generate said user interface description for compatibility with an operating system of said at least one remote computing device.

28. A telemetry interface module according to claim 19, wherein said user interface description is a markup language file.

29. A telemetry interface module according to claim 19, wherein:
said at least one remote computing device renders a user interface in response to said user interface description; and
said communication element being further configured to receive input data from said at least one remote computing device in response to manipulation of said user interface.

30. A telemetry interface module according to claim 29, further comprising second processing logic in communication with said communication element, said second processing logic being configured to generate, in response to said input data, at least one programming command for said IMD.

31. A telemetry interface module according to claim 30, wherein said telemetry element is configured to provide said at least one programming command to said IMD.

32. A telemetry interface module according to claim 29, further comprising second processing logic in communication with said communication element, said second processing logic being configured to generate, in response to said input data, an updated user interface description for said remote computing device.

33. A telemetry system for communicating information between a data source and a destination computer system, said telemetry system comprising:
an implantable medical device (“IMD”); and
a telemetry interface module comprising a telemetry element configured to communicate with said IMD and to receive data from said IMD, said telemetry interface module generating a user interface description in response to said data, and said telemetry interface module providing said user interface description to a remote computing device.

34. A telemetry system according to claim 33, further comprising said remote computing device.

35. A telemetry system according to claim 34, wherein said telemetry interface module generates said user interface description in response to operating characteristics of said remote computing device.

36. A telemetry system according to claim 34, wherein said telemetry interface module communicates with said remote computing device using at least one standardized data communication protocol.

37. A telemetry system according to claim 34, wherein said remote computing device is configured to receive said user interface description and to render a user interface in response to said user interface description.

38. A telemetry system according to claim 33, wherein said telemetry interface module is further configured to receive input data from said remote computing device and, in response to said input data, generate at least one programming command for said IMD.

39. A telemetry system according to claim 38, wherein said telemetry interface module is further configured to communicate said at least one programming command to said IMD.

40. A telemetry system for communicating information between a data source and a destination computer system, said telemetry system comprising:
a remote computing device; and
a telemetry interface module comprising a telemetry element configured to communicate with an implantable medical device (“IMD”) and to receive data from said IMD, said telemetry interface module generating a user interface description in response to said data, and said telemetry interface module providing said user interface description to said remote computing device.

41. A telemetry system according to claim 40, further comprising said IMD, said IMD supporting wireless communication with said telemetry interface module.

42. A telemetry system according to claim 41, wherein said telemetry interface module is further configured to generate at least one programming command for said IMD.

43. A telemetry system according to claim 42, wherein said telemetry interface module is further configured to communicate said at least one programming command to said IMD.

44. A telemetry system according to claim 40, wherein said telemetry interface module generates said user interface description in response to operating characteristics of said remote computing device.

45. A telemetry system according to claim 40, wherein said telemetry interface module communicates with said remote computing device using at least one standardized data communication protocol.

46. A telemetry system according to claim 40, wherein said remote computing device is configured to receive said user interface description and to render a user interface in response to said user interface description.

47. A telemetry system according to claim 46, wherein said telemetry interface module is configured to receive input data in response to manipulation of said user interface.

48. A telemetry system according to claim 46, wherein said telemetry interface module is further configured to generate, in response to said input data, at least one programming command for said IMD.

49. A method for communicating information between an implantable medical device “(IMD”) and a destination computer system, said method comprising:
receiving data at a telemetry interface module, said data originating from said IMD;
said telemetry interface module generating a user interface description in response to said data;
said telemetry interface module providing said user interface description to a remote computing device;
said telemetry interface module receiving IMD programming data originating from said remote computing device;
said telemetry interface module generating, in response to said IMD programming data, at least one programming command for said IMD; and
said telemetry interface module providing said at least one programming command to said IMD.

50. A method according to claim 49, wherein said telemetry interface module generates said user interface description in response to operating characteristics of said remote computing device.

51. A method according to claim 49, wherein said telemetry interface module communicates with said remote computing device in accordance with at least one standardized data communication protocol.

52. A method according to claim 49, further comprising:
said remote computing device receiving said user interface description; and
said remote computing device rendering a user interface in response to said user interface description.

53. A method according to claim 52, further comprising generating said IMD programming data in response to manipulation of said user interface.
